(19) United States
(12) Patent Application Publication
(10) Pub. No.: US 2013/0018244 A1
(43) Pub. Date: Jan. 17, 2013

(54) Budget Your Weight System

(71) Applicants: Massoud Kazemzadeh, Clara City, MN (US); Roshan Kazemzadeh, Clara City, MN (US)

(72) Inventors: Massoud Kazemzadeh, Clara City, MN (US); Roshan Kazemzadeh, Clara City, MN (US)

(73) Assignee: Massoud Kazemzadeh, Clara City, MN (US)

(21) Appl. No.: 13/184,212

(22) Filed: Jul. 15, 2011

(51) Int. Cl. A61B 5/145; G06F 17/30; G06F 19/00; A61B 5/01; A61B 5/021; A61B 5/00; G06F 15/173; A61B 5/024

(52) U.S. Cl. 600/365; 709/224; 707/705; 600/508; 600/549; 600/485; 600/309; 702/19; 707/E17.005

(57) ABSTRACT

A system and method of monitoring a person's health information is disclosed. The method comprises the steps of providing a computer program algorithm accessible by a computer processing device and providing at least one monitor in electronic communication with the computer processing device wherein the monitor is configured to transmit the person's physical data to the computer processing device, and the algorithm is configured to store the physical data, average the physical data over one or more predetermined periods of time, and report the physical data in one or more predetermined formats. The computer processing device may be a cellular telephone, tablet computer, laptop computer, desktop computer, server, or networked computing device. The monitor may be any one of a pedometer, a heart rate monitor, a body temperature monitor, a blood glucose monitor, a blood pressure monitor, or a monitor configured to collect physical data from one or more of a person's body fluids. In some embodiments, the method is used to monitor a person's caloric balance, which may be computed or estimated.

### BACKGROUND

Obesity and diabetes are becoming an epidemic in the United States as well as worldwide. A majority of the people with whom we have spoken have been or are now on some sort of specialty diet or meal plan where the consumption of various foods are monitored closely in order to keep the weight of the individual from increasing or to eliminate any allergens. It is reported by the United States Center for Disease Control (CDC) that 63% of the US population is either obese or overweight. They predict that by the year 2015, 20% of Americans will have diabetes in the form of Type 1 and Type 2. With this alarming increase in the rate of disease among the general population, the need for individuals to keep a close eye on calorie intake and expenditure is becoming more and more necessary.

Most of the foods we eat are carbohydrates. Carbohydrates are turned into sugar in the blood for our bodies to use as a source of energy. Insulin, a hormone produced by the pancreas, helps move sugar form our blood into the cells of our bodies. If your body doesn't make enough insulin or if the insulin doesn't work the way it should, blood sugar cannot get into your cells. Instead, it stays in your blood, raising your blood sugar level. If your body does produce insulin, or if you inject it into your body, it will convert the sugar into energy. If you do not need the energy, the sugar is converted into fat and reserved as stored energy resulting in weight gain.

Here is the danger for diabetics: when too much sugar stays in your blood for a long time, it can damage blood vessels and nerves, resulting in irreversible damage to the very inner structure of your body function. It is, therefore, important to keep your blood sugar controlled. This can usually be done by eating nutrition-dense foods that are digested and absorbed into the blood stream slowly, thus reducing the spike in sugar concentration in the blood. Eating the right foods is the first and most important step in the road to recovery for obesity and diabetes. Foods with little or no sugar that are high in protein and with significant fiber are ideal foods because they are nutrition dense and they digest slowly. Sugar and starches break down to simple sugar and enter the blood stream very quickly, usually in 3-10 minutes, depending on type and source. Protein and fat take between 3-4 hours to enter the blood stream. Although fats take a long time to be digested, they are very high in calories (1 g of fat=9 calories while 1 g of protein or sugars=4 calories).

Choosing your food wisely can help you feel better from day to day, lose weight if you need to, and lower your risk of heart disease, stroke, and other problems caused by diabetes. Vegetarian foods are usually lower in fat and have a very healthy nutritional profile with ample amounts of fiber, protein, vitamins and minerals. Dark fruits such as berries are high in fiber. Dark leafy salads greens are in rich fiber and in iron. Vegetarian based foods such as beans and peas are usually high in protein and fiber, but low in fat. They can deliver as satisfying a taste profile as the animal based proteins. A healthy diet means you reduce your portions to about ½ of your fist, but eat more frequently such as 5 times per day with up to 64 oz. of liquid over the day. This allows your digestive system to continuously release small amounts of sugar into your blood stream and prevents you from having spikes in your blood sugar levels, providing a consistent energy needed to enjoy every hour of the day. By increasing your protein and fiber intake you can also reduce the total daily calorie intake without feeling hungry. You will lose weight and your diabetes will become easier to control perhaps to the point of being able to totally reverse this disease.

Knowing the type of ingredients in your food, but not knowing the calories it contains, represents another major problem in weight gain. Without calorie count, any effort to reduce one's weight becomes frustrating and is not sustainable. For a diabetic, checking one's blood sugar level is the best way to keep the disease under control however knowing how many calories one has consumed at any given time and how many calories have been expended due to activity, gives a better control of how much insulin and or exercise one needs to make the calorie balance of the day to the desired number. It is with this scenario as well as for those who are trying to watch their daily calorie intake and expenditure for general weight management that budgeting one's weight becomes a very applicable solution.

Various diets and diet meals today work on the concept that they need to provide the consumer with the right food to eat. On these plans, one can only eat what the plan provides whether the food palatable or not. No freedom is given to the dieter to choose her own food based on her knowledge of calories. Yet it is the excess calories beyond what is being expended that causes increase in total weight. By utilizing a “budget your weight” program, one can easily see at any time of the day the approximate total calorie count for throughout the day as well as other health information. On a daily basis, one may not find this information as crucial as it is over a period of time, e.g., a week or a month. One can see the accumulated history of health-related data, including calories in verses calories out and can understand why one is losing or gaining weight.

It is reported that Americans gain one pound of weight per year, which leads to the additional use of over 85 million gallons of petroleum to carry the excess weight around. Since this weight gain is slow and not noticeable, the demand for a good way to budget and balance one's calories on daily basis becomes the key to solve this problem in conjunction to weight loss/management plans, meal plans and various diets.

### SUMMARY

The budget-your-weight system is a method of monitoring a person's health information comprising the steps of providing a computer program algorithm accessible by a computer processing device and providing at least one monitor in electronic communication with the computer processing device wherein the monitor is configured to transmit the person's physical data to the computer processing device, and the algorithm is configured to store the physical data, average the physical data over one or more predetermined periods of time, and report the physical data in one or more predetermined formats. The computer processing device may be a cellular telephone, tablet computer, laptop computer, desktop computer, server, or networked computing device. The monitor may be any one of a pedometer, a heart rate monitor, a body temperature monitor, a blood glucose monitor, a blood pressure monitor, or a monitor configured to collect physical data from one or more of a person's body fluids.

In another embodiment, the budget-your-weight system is a method of monitoring a person's caloric balance comprising the steps of providing a computer program algorithm accessible by a computer processing device, providing a monitor in electronic communication with the computer processing device wherein the monitor is configured to transmit the person's physical exertion data to the computer processing device, and providing a user interface on the computer processing device wherein the user interface is configured to collect the person's food consumption data wherein the algorithm is configured to convert the physical exertion data into a caloric output equivalent, convert the food consumption data into a caloric input equivalent, and compute a caloric balance for the person from the caloric output equivalent and the caloric input equivalent and display the caloric balance through the user interface. The method may further comprise providing at least one library of caloric input or output equivalent data accessible by the algorithm. In this embodiment, the computer processing device may be a cellular telephone, tablet computer, laptop computer, desktop computer, server, or networked computing device. The monitor may be any one of a pedometer, a heart rate monitor, a body temperature monitor, a blood glucose monitor, a blood pressure monitor, or a monitor configured to collect physical data from one or more of a person's body fluids.

The food consumption data may include one or more of serving size, calories, sodium, and fiber. The method may further comprise providing a database in a storage medium configured to store one or more of the physical exertion data, the caloric output equivalent, the food consumption data, the caloric input equivalent, and the caloric balance, and the user interface may be configured to access the database. This embodiment may further provide a second user interface configured to access the database through a computer network, and the second user interface may be a website.

### DESCRIPTION OF THE DRAWINGS

FIG. 1 is a schematic representation of the budget-your-weight system.

### DETAILED DESCRIPTION

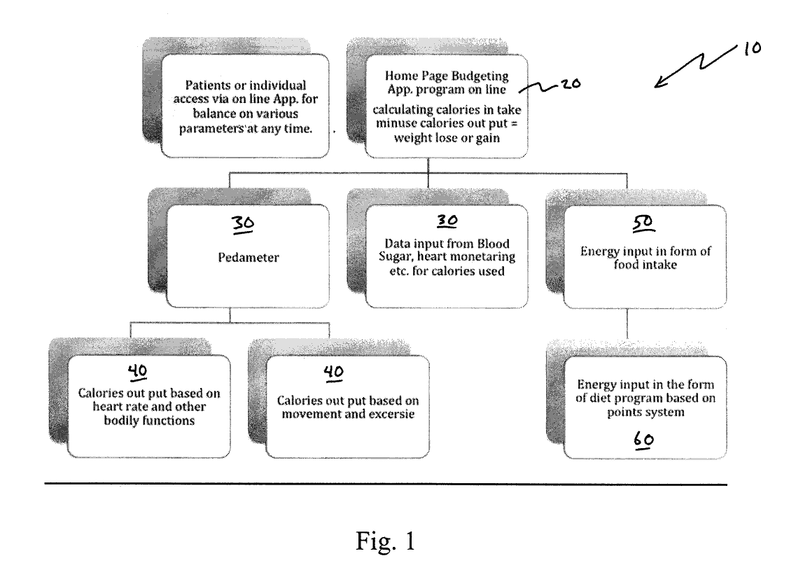

As shown in FIG. 1, the budget-your-weight system 10 comprises a computer program algorithm 20 running on a computer processing device. The algorithm 20 and computer processing device are configured to accept a person's physical data and food consumption data as inputs, store the data, and compute or configure the data inputs into useful outputs such as health trend information or caloric trend information over predetermined periods of time.

The algorithm 20 may be configured to run in any computing environment such as a server, network or “cloud” storage, mobile storage, such as a flash drive, personal computer, or mobile computing device such as a tablet computer or a mobile telephone. In some embodiments, the algorithm 20 may be networked to one or more databases configured to store the person's data or aggregate data from multiple users. In some embodiments, the person using the algorithm 20 is identified by a unique identifier, such as a user name, and the person's access to the algorithm may be controlled by any means, such as a password associated with the user name.

The algorithm 20 is configured to acquire the person's physical data as inputs either manually through a user interface on the computer processing device or from one or more monitors 30 or monitoring devices such as a pedometer, heart rate monitor, body temperature monitor, blood glucose monitor, or blood pressure monitor. In some embodiments, more than one monitor 30 may be used to collect the person's physical data. The algorithm 20 may be configured to collect raw data from the one or more monitors 30, such as steps from a pedometer, pulse or heart rate from a heart rate monitor, body temperature from a body temperature monitor, blood sugar from a blood glucose monitor, or blood pressure from a blood pressure monitor. In some embodiments, the algorithm 20 may convert the raw data into information regarding exercise or daily activities, such as caloric output data 40. In some embodiments, either or both of the raw data or caloric output data may be estimates. In some embodiments, the algorithm 20 may be zeroed out at predetermined intervals, such as daily or weekly, in order to begin each day or week with a fresh start.

In some embodiments, the algorithm 20 is further configured to acquire the person's food consumption data 50, which may be manually entered through a user interface on the computer processing device or in an automated fashion, for instance through digital recognition of food photographed by the user. The algorithm 20 may be configured to convert the food consumption data into calorie intake data, and in some embodiments either or both of the food consumption data or the calorie intake data may be estimates. In some embodiments, the difference between the caloric output data and caloric intake is displayed as a calorie balance in negative calories or positive calories for a predetermined period of time such as hourly, daily, weekly, monthly, or yearly. In some embodiments, certain amounts of calories spent in recurring activities, such as while the person is at rest or asleep, can be programmed into the algorithm 20 to be added automatically.

In embodiments that compute a calorie balance, if the result of the balance is in the form of positive calories, the user may need to expend energy by walking, running, or other exercise to bring the calorie balance to negative for a particular period of time. In some embodiments, the program may be configured to suggest a number of options that one can do to bring the calorie balance to negative. For example, the user may need to walk vigorously for twenty minutes equaling one hundred calories burned. The calorie values for most common daily activities and exercise are reported by the health and fitness industry. These values can be contained in a library 60 electronically accessible by the algorithm 20 to be used as suggestions to the user for budgeting his or her calorie balance.

Most pre-packaged foods and diet programs contain nutritional panels containing food data such as serving size. Nutrition panels contain much information, which can be useful for reporting to the user at any time of the day. Consumption of total calories and the calorie balance for a given point in time can be very essential information for the user. Simultaneous reporting of other nutritional intake, such as total sodium, protein, fat, fiber and sugar, can also allow the individual to make better food choices for the remaining period of the day. In some embodiments, the algorithm 20 may be configured to gather food nutrition data from nutrition panels through photographic imaging of the panel, a universal product code, a quick response code, or the like.

Budgeting one's weight is a great source of information for the user knowing exactly how many calories he or she has consumed or needs to consume in order to reach designated calorie goals. In some embodiments, the algorithm 20 may be configured to keep records of historical information of calorie balances as well as other data, and may be configured to display such information for configurable intervals, such as hourly, daily, monthly, or yearly. This information can also be crucial for a medical professional to monitor a patient's eating and exercise habits for total calorie consumption. It can also show a physician, dietitian or nutritionist what other nutrients the patient is consuming, such as sodium and fiber, and in what quantities.

Since almost all food products contain a nutritional panel, in some embodiments the algorithm 20 is configured to access a library 60 of food products or food nutrition data, so that the user may input the serving size that he or she is consuming in order to get the total values from the nutrition panel for the given period. In some embodiments, the user may save nutrition information for a new food into the library 60 for future calculation. The user then needs to input how many servings of a given product they have consumed at a given time. The calorie balance as well as all other information may be displayed on a user interface, such as the screen of a personal computer or smart phone, for the user to monitor his or her caloric balance, as well as other health information.

In some embodiments, food nutrition information and a library 60 of nutritional panels can be programmed into a website or database which may be accessible to every user of the algorithm 20 with an appropriate registration and password identifying the user and his or her account. Since this program can work with any diet system whether for gaining weight or for losing weight, it renders itself to partner up with a number of diet plans for the benefit of the consumer. It also allows the consumer to have better control over eating habits and daily activities for exercise for the output of energy.

Since in some embodiments where the results are based on a caloric balance, the algorithm 20 may be further customized with additional personal data, such as the individual user's height, initial weight, personal activity or profession, as well as any other relevant health information, such as the presence of a serious disease like diabetes, hyperglycemia, and the like that may relevant to the health or metabolism of the user. In some embodiments, the algorithm 20 may be configured to simply approximate a daily calorie balance for individuals who wish to lose or gain weight.

In some embodiments, the algorithm 20 may also be configured to display particular data gathered from specialized monitors. For instance, in some embodiments, the algorithm 20 may be configured to assess blood chemistry and physical data through a blood monitor, in which a user may see such data as white blood cell counts and protein counts. Other specialty monitors may be configured to gather data from other body fluids such as saliva or urine.

One exemplary embodiment of the budget-your-weight system is a method of monitoring a person's health information comprising the steps of (a) providing a computer program algorithm accessible by a computer processing device; (b) providing at least one monitor in electronic communication with the computer processing device wherein the monitor is configured to transmit the person's physical data to the computer processing device; (c) wherein the algorithm is configured to store the physical data; and (d) wherein the algorithm is further configured to average the physical data over one or more predetermined periods of time and report the physical data in one or more predetermined formats. This method may further comprise the computer processing device being a cellular telephone, tablet computer, laptop computer, desktop computer, server, or networked computing device. In addition, this method may further comprise the at least one monitor being one or more of a pedometer, a heart rate monitor, a body temperature monitor, a blood glucose monitor, a blood pressure monitor, or a monitor configured to collect physical data from one or more of the person's body fluids.

Another exemplary embodiment of the budget-your-weight system 10 is a method of monitoring a person's caloric balance comprising the steps of: (a) providing a computer program algorithm accessible by a computer processing device; (b) providing a monitor in electronic communication with the computer processing device wherein the monitor is configured to transmit the person's physical exertion data to the computer processing device; (c) providing a user interface on the computer processing device wherein the user interface is configured to collect the person's food consumption data; (d) wherein the algorithm is configured to convert the physical exertion data into a caloric output equivalent; (e) wherein the algorithm is configured to convert the food consumption data into a caloric input equivalent; and (f) wherein the algorithm is configured to compute a caloric balance for the person from the caloric output equivalent and the caloric input equivalent and display the caloric balance through the user interface. This method may further comprise providing at least one library of caloric input or output equivalent data accessible by the algorithm. Further, in this embodiment, the computer processing device may be a cellular telephone, tablet computer, laptop computer, desktop computer, server, or networked computing device. Additionally, this method may further comprise the at least one monitor being one or more of a pedometer, a heart rate monitor, a body temperature monitor, a blood glucose monitor, a blood pressure monitor, or a monitor configured to collect physical data from one or more of the person's body fluids. In this embodiment, the food consumption data may include one or more of serving size, calories, sodium, and fiber. The embodiment may also further comprise providing a database in a storage medium configured to store one or more of the physical exertion data, the caloric output equivalent, the food consumption data, the caloric input equivalent, and the caloric balance wherein the user interface is configured to access the database. Further, a second user interface configured to access the database through a computer network may be provided in this embodiment, and the second user interface may be a website.

While the system 10 has been described in reference to some exemplary embodiments, these embodiments are not limiting and are not necessarily exclusive of each other, and it is contemplated that particular features of various embodiments may be omitted or combined for use with features of other embodiments while remaining within the scope of the invention.

We claim:

1. A method of monitoring a person's health information comprising the steps of:
(a) providing a computer program algorithm accessible by a computer processing device;
(b) providing at least one monitor in electronic communication with the computer processing device wherein the monitor is configured to transmit the person's physical data to the computer processing device;
(c) wherein the algorithm is configured to store the physical data; and
(d) wherein the algorithm is further configured to average the physical data over one or more predetermined periods of time and report the physical data in one or more predetermined formats.

2. The method of claim 1, further comprising:
wherein the computer processing device is a cellular telephone.

3. The method of claim 1, further comprising:
wherein the monitor is a pedometer.

4. The method of claim 1, further comprising:
wherein the monitor is a heart rate monitor.

5. The method of claim 1, further comprising:
wherein the monitor is a body temperature monitor.

6. The method of claim 1, further comprising:
wherein the monitor is a blood glucose monitor.

7. The method of claim 1, further comprising:
wherein the monitor is a blood pressure monitor.

8. The method of claim 1, further comprising:
wherein the monitor is configured to collect physical data from one or more of the person's body fluids.

9. A method of monitoring a person's caloric balance comprising the steps of:
(a) providing a computer program algorithm accessible by a computer processing device;
(b) providing a monitor in electronic communication with the computer processing device wherein the monitor is configured to transmit the person's physical exertion data to the computer processing device;
(c) providing a user interface on the computer processing device wherein the user interface is configured to collect the person's food consumption data;
(d) wherein the algorithm is configured to convert the physical exertion data into a caloric output equivalent;
(e) wherein the algorithm is configured to convert the food consumption data into a caloric input equivalent; and
(f) wherein the algorithm is configured to compute a caloric balance for the person from the caloric output equivalent and the caloric input equivalent and display the caloric balance through the user interface.

10. The method of claim 9, further comprising:
providing at least one library of caloric input or output equivalent data accessible by the algorithm.

11. The method of claim 9, further comprising:
wherein the computer processing device is a cellular telephone.

12. The method of claim 9, further comprising:
wherein the monitor is a pedometer.

13. The method of claim 9, further comprising:
wherein the monitor is a heart rate monitor.

14. The method of claim 9, further comprising:
wherein the monitor is a body temperature monitor.

15. The method of claim 9, further comprising:
wherein the monitor is a blood glucose monitor.

16. The method of claim 9, further comprising:
wherein the monitor is a blood pressure monitor.

17. The method of claim 9, further comprising:
wherein the monitor is configured to collect physical data from one or more of the person's body fluids.

18. The method of claim 9, further comprising:
wherein the food consumption data includes one or more of serving size, calories, sodium, and fiber.

19. The method of claim 9, further comprising:
providing a database in a storage medium configured to store one or more of the physical exertion data, the caloric output equivalent, the food consumption data, the caloric input equivalent, and the caloric balance; and
wherein the user interface is configured to access the database.

20. The method of claim 19, further comprising:
providing a second user interface configured to access the database through a computer network.

21. The method of claim 20, further comprising:
wherein the second user interface is a website.
